(19) United States
(12) Patent Application Publication
(10) Pub. No.: US 2023/0320831 A1
(43) Pub. Date: Oct. 12, 2023

(54) IMPLANTABLE INFLATABLE DEVICE

(71) Applicant: Boston Scientific Scimed, Inc., Maple Grove, MN (US)

(72) Inventors: Thomas Sinnott, Enniscorthy (IE); Eduardo Marcos Larangeira, Cork City (IE); Brian P. Watschke, Minneapolis, MN (US); Daragh Nolan, Via Youghal (IE)

(21) Appl. No.: 18/190,382

(22) Filed: Mar. 27, 2023

Related U.S. Application Data
(60) Provisional application No. 63/362,156, filed on Mar. 30, 2022.

(51) Int. Cl. A61F 5/41; A61F 2/00

(52) U.S. Cl. CPC A61F 2/004; A61F 5/41; A61F 2005/415

(57) ABSTRACT

An implantable inflatable device includes an elongate member having a storage portion and an inflatable portion and a pump assembly operatively coupled to the elongate member and configured to transfer fluid from the storage portion of the elongate member to the inflatable portion of the elongate member.

### CROSS-REFERENCE TO RELATED APPLICATION

This application claims priority to U.S. Provisional Pat. Application No. 63/362,156, filed on Mar. 30, 2022, entitled “IMPLANTABLE INFLATABLE DEVICE”, the disclosure of which is incorporated by reference herein in its entirety.

### TECHNICAL FIELD

This disclosure relates generally to bodily implants, and more specifically to bodily implants including an inflatable member and a pump.

### BACKGROUND

Implantable inflatable devices often include one or more pumps that regulate a flow of fluid between different portions of the implantable device to provide for inflation and deflation of one or more fluid fillable implant components of the device. For example, some implantable inflatable devices include an inflatable member and a pump or pump assembly. In some implantable inflatable devices, each of the components of the device are disposed or implanted into different portions of the body of the patient. Accordingly, to eliminate the need to place portions of the implant in multiple portions of the body of the patient, it may be desirable to provide an implantable inflatable device with a limited number of components.

### SUMMARY

According to an aspect, an implantable inflatable device includes an elongate member having a storage portion and an inflatable portion and a pump assembly operatively coupled to the elongate member and configured to transfer fluid from the storage portion of the elongate member to the inflatable portion of the elongate member.

In some embodiments, the storage portion is fluidically coupled to the inflatable portion, the elongate member includes a flow control member disposed between the storage portion and the inflatable portion. In some embodiments, the storage portion is fluidically coupled to the inflatable portion, the elongate member includes a flow restrictor or a valve disposed between the storage portion and the inflatable portion.

In some embodiments, the elongate member is a first elongate member, the device further includes a second elongate member having a storage portion and an inflatable portion, the pump assembly being operatively coupled to the second elongate member and configured to transfer fluid from the storage portion of the second elongate member to the inflatable portion of the second elongate member.

In some embodiments, the elongate member is a first elongate member, the device further includes a second elongate member having an inflatable portion, the pump assembly being operatively coupled to the second elongate member and configured to transfer fluid from the storage portion to the inflatable portion of the second elongate member.

In some embodiments, the elongate member includes an adjustable portion, the storage portion being disposed between the adjustable portion and the inflatable portion. In some embodiments, the elongate member includes an adjustable portion, the adjustable portion being configured to be cut to adjust the size of the adjustable portion. In some embodiments, the elongate member includes an adjustable portion, the adjustable portion including visual indicia. In some embodiments, the elongate member includes an adjustable portion, the adjustable portion including a plurality of pieces configured to be removably coupled to each other.

In some embodiments, the pump assembly is operatively coupled to the elongate member via a tubular member. In some embodiments, the pump assembly is operatively coupled to the storage portion of the elongate member via a single tubular member.

In some embodiments, the elongate member is a first elongate member, the device further including a second elongate member, the pump assembly being operatively coupled to the first elongate member and the second elongate member via a Y shaped tubular member.

In some embodiments, the elongate member is configured to be disposed within a penis of a patient. In some embodiments, the elongate member and the pump assembly are configured to be disposed within a pelvic region of a patient.

In some embodiments, the pump assembly is configured to be operatively coupled to a controller.

According to an aspect, an implantable inflatable device, comprising an elongate member having an inflatable portion; a pump assembly operatively coupled to the elongate member via a tubular member, the tubular member being configured to house fluid, the pump assembly being configured to transfer fluid from the tubular member to the inflatable portion of the elongate member.

In some embodiments, the tubular member includes a first portion having a first size and a second portion having a second size, the second size being larger than the first size. In some embodiments, the tubular member includes a first portion having a first size, a second portion having a second size, and a third portion having a third size, the second size being larger than the first size and larger than the third size, the second portion being disposed between the first portion and the third portion.

In some embodiments, the elongate member is a first elongate member, the device further comprising a second elongate member.

In some embodiments, the pump assembly is configured to be wirelessly coupled to a controller.

### BRIEF DESCRIPTION OF THE DRAWINGS

FIG. 1 is a schematic illustration of an implantable inflatable device according to an aspect.

FIGS. 2-3 illustrate an implantable inflatable device according to an aspect.

FIGS. 4-9 illustrate implantable inflatable devices according to aspects.

FIGS. 10-13 illustrate elongate members of implantable inflatable devices according to aspects.

FIGS. 14-16 illustrate an elongate member according to an aspect.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

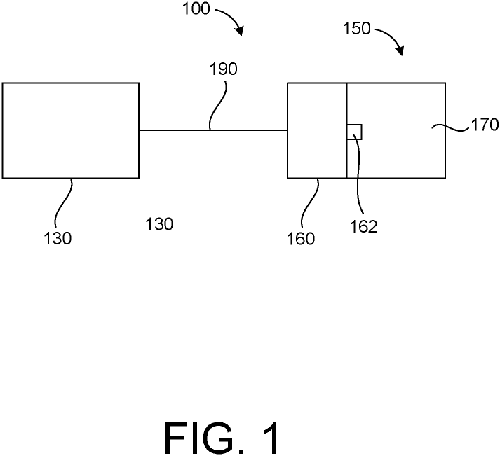

FIG. 1 is a schematic illustration of an implantable inflatable device 100. The device 100 includes a pump assembly 130 and an elongate member 150. The elongate member includes a storage portion 160 and an inflatable portion 170. The storage portion 160 of the elongate member 150 is configured to house fluid. The elongate member 150 is operatively coupled to the pump assembly 130 via connection member 190. Connection member 190 may be a tubular member such as a kink resistant tubing (KRT). In other implementations, the elongate member 150 is operatively coupled to the pump assembly 130 via a different mechanism.

The implantable inflatable device 100 may be configured to be implanted into a body of a patient or user. In some embodiments, the inflatable device 100 may be implanted into a pelvic region of the patient. For example, in some embodiments, the implantable inflatable device 100 is a penile implant. In such embodiments, the elongate member 150 may be implanted into the corpus cavernosae of the patient or user and the pump assembly 130 may be implanted into a portion of the body of the user, such as a scrotum or an abdomen of the user. In other embodiments, the implantable inflatable device 100 is implanted into a different portion of the body of the patient and/or is implanted for a different purpose. For example, in some embodiments, the implantable inflatable device 100 may be an artificial sphincter, such as an artificial urinary sphincter.

The pump assembly 130 may include a pump or more than one pump that is configured pump fluid from the storage portion 160 of the elongate member 150 into the inflatable portion 170 of the elongate member 150 during an inflation cycle. In some examples, the pump or pumps maybe be manually controlled by the user or may be mechanically and/or programmatically controlled by a controller.

The inflation portion 170 of the elongate member 150 may be capable of expanding upon the injection of fluid into a cavity of the inflation portion 170 of the elongate member 150. For instance, upon injection of the fluid into the inflation portion 170, the inflation portion 170 of the elongate member 150 may increase its length and/or width, as well as increase its rigidity. In some examples, the elongate member 150 may include a pair of inflatable cylinders or at least two cylinders, e.g., a first cylinder member and a second cylinder member. The volumetric capacity of the elongate member 150 may depend on the size of the inflatable cylinders.

In the illustrated embodiment, the elongate member 150 includes a fluid flow restrictor 162. The fluid flow restrictor 162 is configured to regulate the flow of fluid between the storage portion 160 and the inflatable portion 170. The fluid flow restrictor 162 is disposed between the storage portion 160 and the inflatable portion 170. In some embodiments, the fluid flow restrictor 162 includes a screen or valve or valve system that allows the fluid to be advanced into the inflatable portion 170 and return to the storage portion 160.

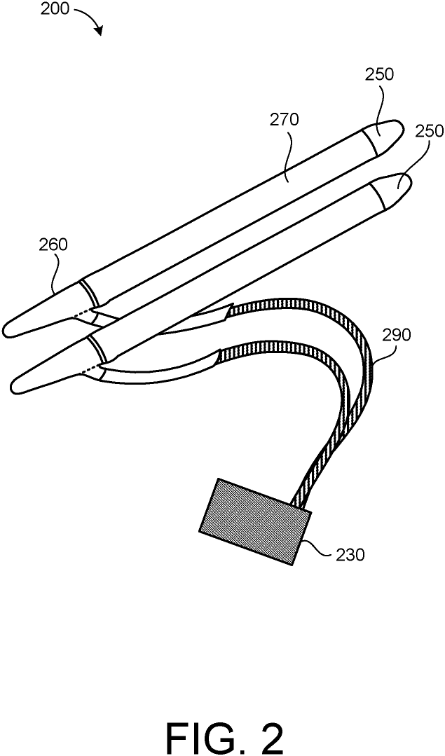

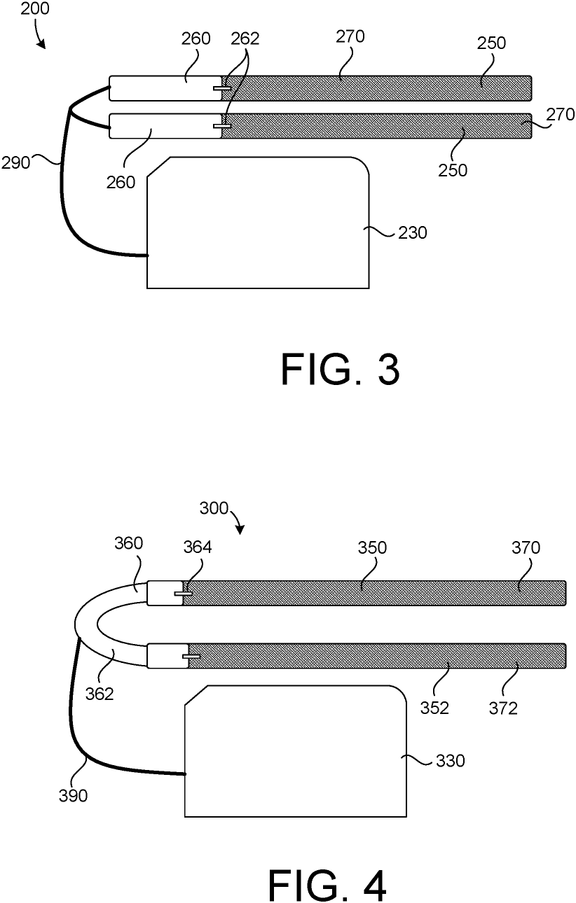

FIGS. 2 and 3 illustrate an inflatable penile prosthesis or device 200. The device 200 includes a pump assembly 230 and an elongate member 250. The elongate member 250 includes a storage portion 260 and an inflatable portion 270. The storage portion 260 of the elongate member 250 is configured to house fluid. The elongate member 250 is operatively coupled to the pump assembly 230 via connection member 290. In the illustrated embodiment, the connection member 290 is a tubular member such as a kink resistant tubing (KRT). In the illustrated embodiment, the connection member 290 is a single tubular member. In other implementations, the elongate member 250 is operatively coupled to the pump assembly 230 via a different mechanism.

In the illustrated embodiment, the device 200 includes two elongate members 250. Each of the elongate members 250 are operatively coupled to the pump assembly 230 via the tubular member 290. In the illustrated embodiment, the tubular member 290 forms a Y shape with the short ends of the Y coupled to the elongate members 250 and the long end of the Y coupled to the pump assembly 230.

The implantable inflatable device 200 may be configured to be implanted into a body of a patient or user. For example, in some embodiments, the elongate member 250 may be implanted into the corpus cavernosae of the patient or user and the pump assembly 230 may be implanted into a portion of the body of the user, such as a scrotum or an abdomen of the user. Accordingly, in the illustrated embodiment, there is a need for placement of the device in only 2 different locations within the body of the patient (rather than a device with more portion or member requiring more than 2 different surgical locations within the body of the patient). In other embodiments, the implantable inflatable device 200 is implanted into a different portion of the body of the patient and/or is implanted for a different purpose. For example, in some embodiments, the implantable inflatable device 200 may be an artificial sphincter, such as an artificial urinary sphincter.

The pump assembly 230 may include a pump or more than one pump that is configured pump fluid from the storage portion 260 of the elongate member 250 into the inflatable portion 270 of the elongate member 250 during an inflation cycle. In some examples, the pump or pumps maybe be manually controlled by the user or may be mechanically and/or programmatically controlled by a controller.

The inflation portion 270 of the elongate member 250 may be capable of expanding upon the injection of fluid into a cavity of the inflation portion 270 of the elongate member 250. For instance, upon injection of the fluid into the inflation portion 270, the inflation portion 270 of the elongate member 250 may increase its length and/or width, as well as increase its rigidity. The volumetric capacity of the elongate member 250 may depend on the size of the inflatable cylinders.

In the illustrated embodiment, the elongate member 250 includes a fluid flow restrictor 262. The fluid flow restrictor 262 is configured to regulate the flow of fluid between the storage portion 260 and the inflatable portion 270. The fluid flow restrictor 262 is disposed between the storage portion 260 and the inflatable portion 270. In some embodiments, the fluid flow restrictor 262 includes a screen or valve or valve system that allows the fluid to be advanced into the inflatable portion 270 and return to the storage portion 260.

In the illustrated embodiment, the elongate member 250 and the pump assembly 230 may be formed with biocompatible materials. In some embodiments, the storage portion 260 of the elongate member 250 can be of a variety of shapes and sizes and may be formed of molded silicone, machined metal, titanium or other implantable material. The inflatable portion 270 may be bonded, molded, or two-shot molded over the storage portion 260.

FIG. 4 illustrates an inflatable penile prosthesis or device 300. The device 300 includes a pump assembly 330 and a first elongate member 350, and a second elongate member 352. The first elongate member 350 includes a storage portion 360 and an inflatable portion 370. The second elongate member 352 includes a storage portion 362 and an inflatable portion 372. The portions of the elongate members 350 and 352 are similarly constructed so for ease, only the elongate member 350 will be discussed in detail. The storage portion 360 of the first elongate member 350 is configured to house fluid. The first elongate member 350 is operatively coupled to the pump assembly 330 via connection member 390.

In the illustrated embodiment, the storage portion 360 of the first elongate member 350 is fluidically coupled to the storage portion 362 of the second elongate member 352. In some embodiments, a single storage portion may be fluidically coupled to the first elongate member and to the second elongate member.

The implantable inflatable device 300 may be configured to be implanted into a body of a patient or user. For example, in some embodiments, the elongate member 350 may be implanted into the corpus cavernosae of the patient or user and the pump assembly 330 may be implanted into a portion of the body of the user, such as a scrotum or an abdomen of the user. Accordingly, in the illustrated embodiment, there is a need for placement of the device in only 2 different locations within the body of the patient (rather than a device with more portion or member requiring more than 2 different surgical locations within the body of the patient). In other embodiments, the implantable inflatable device 300 is implanted into a different portion of the body of the patient and/or is implanted for a different purpose. For example, in some embodiments, the implantable inflatable device 300 may be an artificial sphincter, such as an artificial urinary sphincter.

The pump assembly 330 may include a pump or more than one pump that is configured pump fluid from the storage portion 360 of the elongate member 350 into the inflatable portion 370 of the elongate member 350 during an inflation cycle. In some examples, the pump or pumps maybe be manually controlled by the user or may be mechanically and/or programmatically controlled by a controller.

The inflation portion 370 of the elongate member 350 may be capable of expanding upon the injection of fluid into a cavity of the inflation portion 370 of the elongate member 350. For instance, upon injection of the fluid into the inflation portion 370, the inflation portion 370 of the elongate member 350 may increase its length and/or width, as well as increase its rigidity. The volumetric capacity of the elongate member 350 may depend on the size of the inflatable cylinder.

In the illustrated embodiment, the elongate member 350 includes a fluid flow restrictor 364. The fluid flow restrictor 364 is configured to regulate the flow of fluid between the storage portion 360 and the inflatable portion 370. The fluid flow restrictor 364 is disposed between the storage portion 360 and the inflatable portion 370. In some embodiments, the fluid flow restrictor 364 includes a screen or valve or valve system that allows the fluid to be advanced into the inflatable portion 370 and return to the storage portion 360.

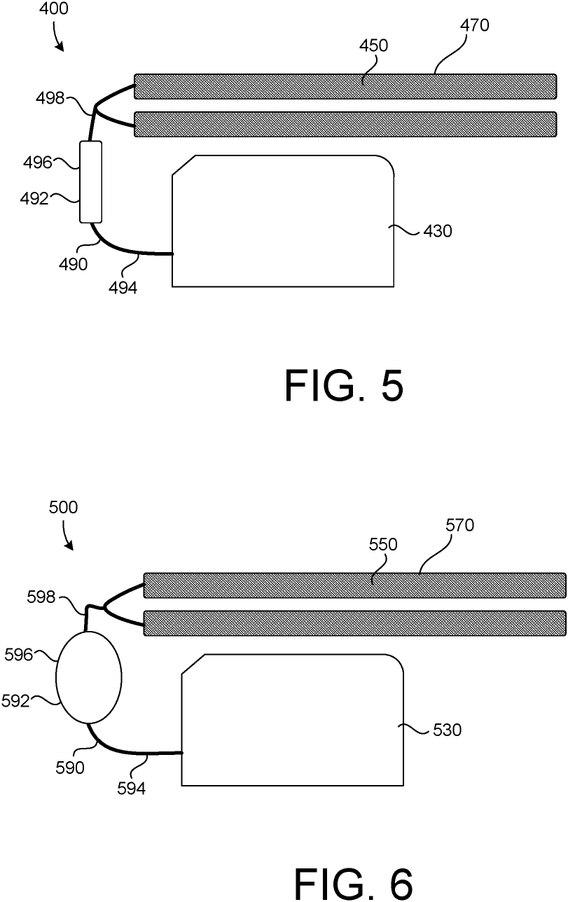

FIG. 5 illustrates an inflatable penile prosthesis or device 400. The device 400 includes a pump assembly 430 and an elongate member 450. The elongate member 450 includes an inflatable portion 470. The elongate member 450 is operatively coupled to the pump assembly 430 via connection member 490. In the illustrated embodiment, the connection member 490 is a tubular member such as a kink resistant tubing (KRT). In the illustrated embodiment, the connection member 490 is a single tubular member. In other implementations, the elongate member 450 is operatively coupled to the pump assembly 430 via a different mechanism.

In the illustrated embodiment, the connection member 490 includes a storage portion 492. The storage portion 492 is disposed between in the elongate member 450 and the pump assembly 430. In the illustrated embodiment, the connection member 490 includes a first portion 494, a second portion 496 (which includes the storage portion 492), and a third portion 498. The second portion 496 is disposed between the first portion 494 and the third portion 498. The first portion 494 has a size, such as a diameter, that is smaller than a size, such as a diameter, of the second portion 496. Similarly, the third portion 498 has a size, such as a diameter, that is smaller than a size, such as a diameter, of the second portion 496. In some embodiments, the first portion 494, the second portion 496, and the third portion 498 are integrally formed. In other embodiments, the portions are formed separately and coupled together using connection members or other connection means.

FIG. 6 illustrates an inflatable penile prosthesis or device 500. The device 500 includes a pump assembly 530 and an elongate member 550. The elongate member 550 includes an inflatable portion 570. The elongate member 550 is operatively coupled to the pump assembly 530 via connection member 590. In the illustrated embodiment, the connection member 590 is a tubular member such as a kink resistant tubing (KRT). In the illustrated embodiment, the connection member 590 is a single tubular member. In other implementations, the elongate member 550 is operatively coupled to the pump assembly 530 via a different mechanism.

In the illustrated embodiment, the connection member 590 includes a storage portion 592. The storage portion 592 is disposed between in the elongate member 550 and the pump assembly 530. In the illustrated embodiment, the connection member 590 includes a first portion 594, a second portion 596 (which includes the storage portion 592), and a third portion 598. The second portion 596 is disposed between the first portion 594 and the third portion 598. The first portion 594 has a size, such as a diameter, that is smaller than a size, such as a diameter, of the second portion 596. Similarly, the third portion 598 has a size, such as a diameter, that is smaller than a size, such as a diameter, of the second portion 596. In some embodiments, the first portion 594, the second portion 596, and the third portion 598 are integrally formed. In other embodiments, the portions are formed separately and coupled together using connection members or other connection means.

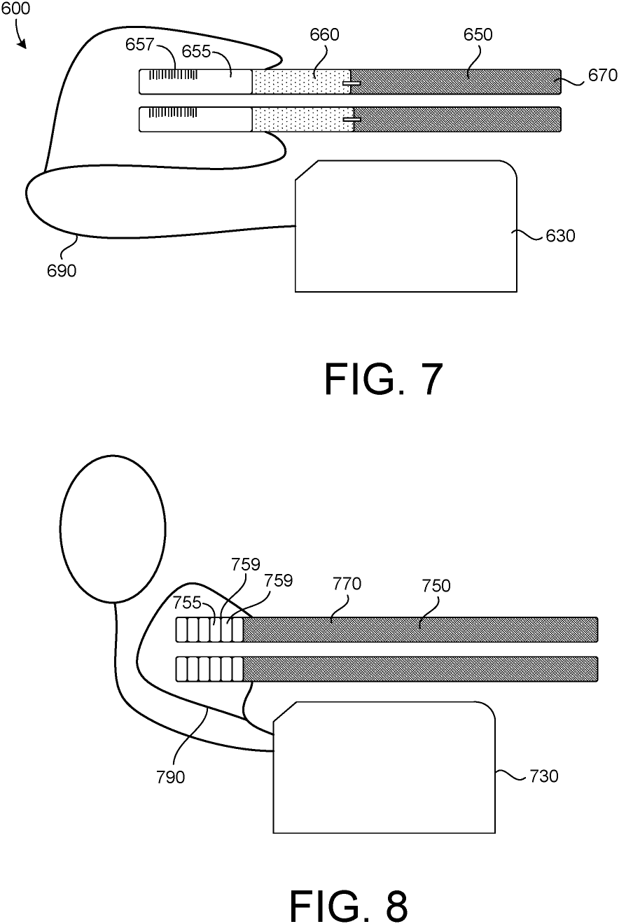

FIG. 7 illustrates an inflatable penile prosthesis or device 600. The device 600 includes a pump assembly 630 and an elongate member 650. The elongate member 650 includes a storage portion 660 and an inflatable portion 670. The storage portion 660 of the elongate member 650 is configured to house fluid. The elongate member 650 is operatively coupled to the pump assembly 630 via connection member 690. In the illustrated embodiment, the connection member 690 is a tubular member such as a kink resistant tubing (KRT). In other implementations, the elongate member 650 is operatively coupled to the pump assembly 630 via a different mechanism.

In the illustrated embodiment, the elongate member 650 includes an adjustable portion 655. The adjustable portion 655 is configured to be adjusted or sized such that it fits within a particular patient. In the illustrated embodiment, the adjustable portion 655 is configured to be cut or trimmed by a physician before or at the time of implanting the device 600 within the body of the patient. In some embodiments, the adjustable portion 655 includes markings such as lines or other visual indicia. The visual indicia may indicate, for example, the length or the size of the device at that location. The adjustable portion 655 is disposed adjacent the storage portion 660 such that the storage portion 660 is disposed between the adjustable portion 655 and the inflatable portion 670. In the illustrated embodiment, the adjustable portion 655 includes markings or indicia (size indications) 657 to facilitate the cutting or sizing of the device by the physician.

FIG. 8 illustrates an inflatable penile prosthesis or device 700. The device 700 includes a pump assembly 730 and an elongate member 750. The elongate member 750 includes an inflatable portion 770. The elongate member 750 is operatively coupled to the pump assembly 730 via connection member 790. In the illustrated embodiment, the connection member 790 is a tubular member such as a kink resistant tubing (KRT). In other implementations, the elongate member 750 is operatively coupled to the pump assembly 630 via a different mechanism.

In the illustrated embodiment, the elongate member 750 includes an adjustable portion 755. The adjustable portion 755 is configured to be adjusted or sized such that it fits within a particular patient. In the illustrated embodiment, the adjustable portion 755 includes a plurality of extenders 759. The plurality of extenders are configured to be removably coupled to one another. Accordingly, a physician, before or at the time of implantation, may couple or decoupled a desired number of extenders to arrive at a desired length of the elongate member 750.

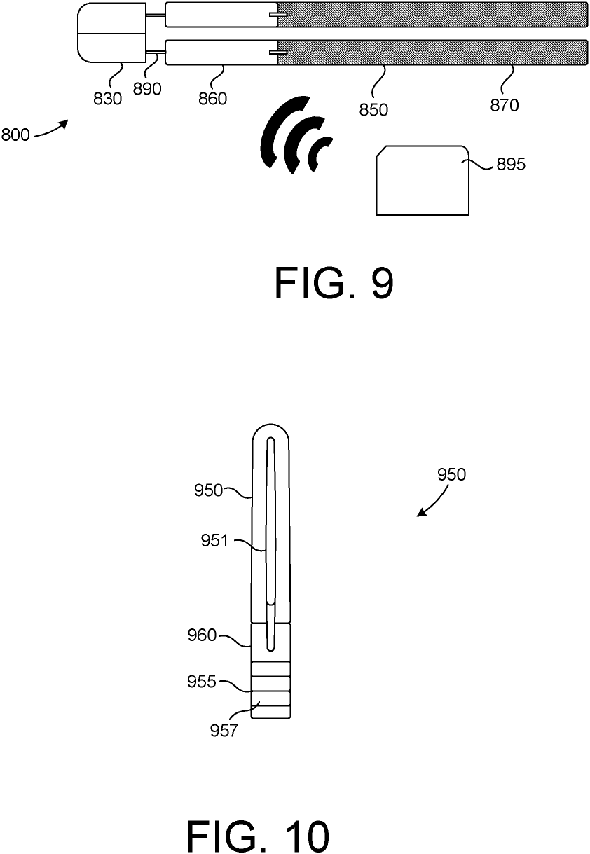

FIG. 9 illustrates an inflatable penile prosthesis or device 800. The device 800 includes a pump assembly 830 and an elongate member 850. The elongate member 850 includes a storage portion 860 and an inflatable portion 870. The storage portion 860 of the elongate member 850 is configured to house fluid. The elongate member 850 is operatively coupled to the pump assembly 830 via connection member 890. In the illustrated embodiment, the connection member 890 is a tubular member such as a kink resistant tubing (KRT). In other implementations, the elongate member 850 is operatively coupled to the pump assembly 830 via a different mechanism.

In the illustrated embodiment, the device 800 includes a controller 895. The controller 895 is configured to control (to activate or deactivate) the pump assembly 830. In some embodiments, the controller 895 is configured to control the pump assembly 830 from a distance. For example, in some embodiments, the controller 895 may be disposed at a different location within the body of the patient and can control the pump assembly 830. In some embodiments, the controller 895 is wirelessly connected to the pump assembly 830. For example, in some embodiments, the controller 895 is wirelessly connected to the pump via radio frequency or another type of wireless technology. In some embodiments, the controller 895 is a hermetically sealed unit and may be coated in a silicone. In some embodiments, the controller 895 may be placed in the scrotum of the patient.

FIG. 10 illustrates an elongate member 950 of an inflatable penile prosthesis or device. The elongate member 950 includes a storage portion 960 and an inflatable portion 970. The storage portion 960 of the elongate member 950 is configured to house fluid. The elongate member 950 may be operatively coupled to a pump assembly via a connection member.

In the illustrated embodiment, the elongate member 950 includes a structure member 951. In the illustrated embodiment, the structure member 951 is disposed within the inflatable portion 970 and is configured to provide structure to the inflatable portion 970. In some embodiments, the structure member 951 is formed of a shape memory alloy. In some embodiments, the structure member is a single strand. In other embodiments, the structure member is a braided wire.

In the illustrated embodiment, the elongate member 950 includes an adjustable portion 955. The adjustable portion 955 is configured to be adjusted or sized such that it fits within a particular patient. In the illustrated embodiment, the adjustable portion 955 is configured to be cut or trimmed by a physician before or at the time of implanting the device 900 within the body of the patient. In the illustrated embodiment, the adjustable portion 955 includes markings or indicia (size indications) 957 to facilitate the cutting or sizing of the device by the physician.

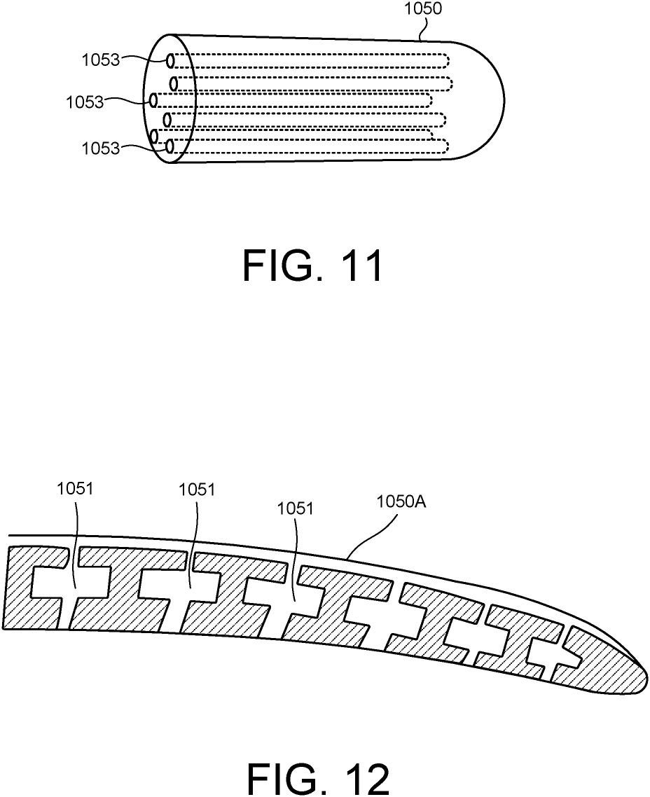

FIG. 11 illustrates an elongate member 1050 of an inflatable penile prosthesis or device. The elongate member 1050 includes a plurality of lumens 1053. The lumens 1053 are configured to receive fluid when the elongate member 1050 is placed in its inflated state.

FIG. 12 illustrates an elongate member 1050A of an inflatable penile prosthesis or device. The elongate member 1050A includes a plurality of internal chambers 1051. The internal chambers 1051 or voids are connected in parallel and are configured to receive fluid to place the elongate member 1050A in an inflated state. The internal chambers 1051 allow for a reduced amount of fluid needed to inflate the elongate member 1050A.

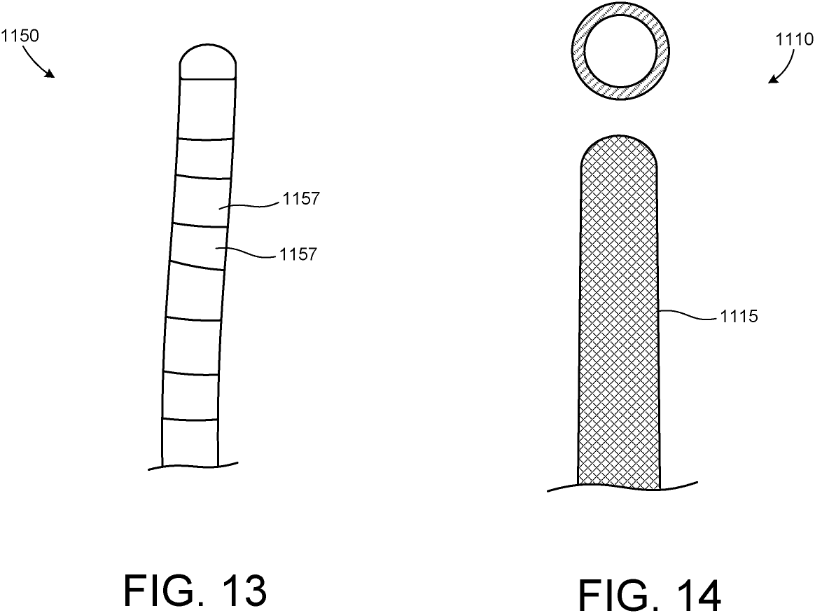

FIG. 13 illustrates an elongate member 1150 of an inflatable penile prosthesis or device. The elongate member 1150 includes a plurality of articulating joints or members 1157. The articulating joints or members 1157 are configured to provide structure to the inflatable portion of the elongate member 1150.

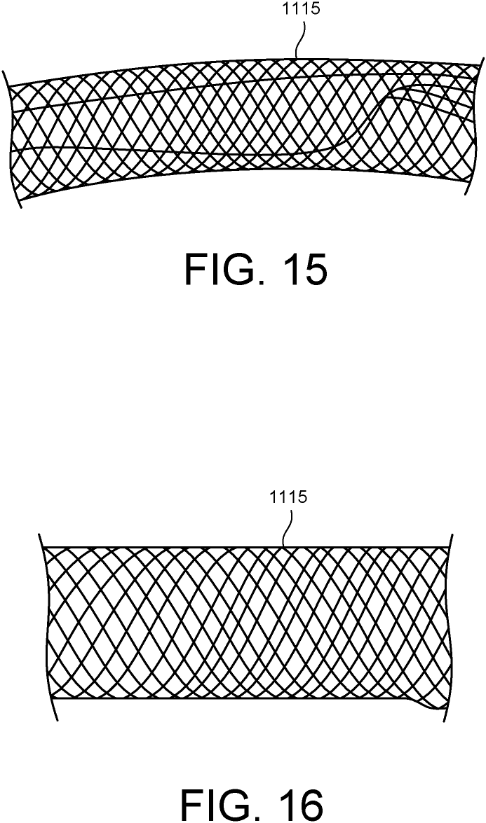

FIGS. 14-16 illustrate an outer portion 1110 of an elongate member according to an embodiment. The outer portion 1110 includes a covering 1115. The covering 1115 if formed of a fabric or a textile. The covering 1115 is formed in a cross-ply pattern. The cross-ply form may provide rigidity as it is expanded. FIG. 15 illustrates the covering 1115 in a deflated or contracted state. FIG. 16 illustrates the covering 1115 is an expanded or inflated state.

While certain features of the described implementations have been illustrated as described herein, many modifications, substitutions, changes, and equivalents will now occur to those skilled in the art. It is, therefore, to be understood that the appended claims are intended to cover all such modifications and changes as fall within the scope of the embodiments.

What is claimed is:

1. An implantable inflatable device, comprising:
an elongate member having a storage portion and an inflatable portion; and
a pump assembly operatively coupled to the elongate member and configured to transfer fluid from the storage portion of the elongate member to the inflatable portion of the elongate member.

2. The implantable inflatable device of claim 1, wherein the storage portion is fluidically coupled to the inflatable portion, the elongate member includes a flow control member disposed between the storage portion and the inflatable portion.

3. The implantable inflatable device of claim 1, wherein the storage portion is fluidically coupled to the inflatable portion, the elongate member includes a flow restrictor or a valve disposed between the storage portion and the inflatable portion.

4. The implantable inflatable device of claim 1, wherein the elongate member is a first elongate member, the device further comprising:
a second elongate member having a storage portion and an inflatable portion, the pump assembly being operatively coupled to the second elongate member and configured to transfer fluid from the storage portion of the second elongate member to the inflatable portion of the second elongate member.

5. The implantable inflatable device of claim 1, wherein the elongate member is a first elongate member, the device further comprising:
a second elongate member having an inflatable portion, the pump assembly being operatively coupled to the second elongate member and configured to transfer fluid from the storage portion to the inflatable portion of the second elongate member.

6. The implantable inflatable device of claim 1, wherein the elongate member includes an adjustable portion, the storage portion being disposed between the adjustable portion and the inflatable portion.

7. The implantable inflatable device of claim 1, wherein the elongate member includes an adjustable portion, the adjustable portion being configured to be cut to adjust a size of the adjustable portion.

8. The implantable inflatable device of claim 1, wherein the elongate member includes an adjustable portion, the adjustable portion including visual indicia.

9. The implantable inflatable device of claim 1, wherein the elongate member includes an adjustable portion, the adjustable portion including a plurality of pieces configured to be removably coupled to each other.

10. The implantable inflatable device of claim 1, wherein the pump assembly is operatively coupled to the elongate member via a tubular member.

11. The implantable inflatable device of claim 1, wherein the pump assembly is operatively coupled to the storage portion of the elongate member via a single tubular member.

12. The implantable inflatable device of claim 1, wherein the elongate member is a first elongate member, the device further comprising:
a second elongate member,
the pump assembly being operatively coupled to the first elongate member and the second elongate member via a Y shaped tubular member.

13. The implantable inflatable device of claim 1, wherein the elongate member is configured to be disposed within a penis of a patient.

14. The implantable inflatable device of claim 1, wherein the elongate member and the pump assembly are configured to be disposed within a pelvic region of a patient.

15. The implantable inflatable device of claim 1, wherein the pump assembly is configured to be operatively coupled to a controller.

16. An implantable inflatable device, comprising:
an elongate member having an inflatable portion;
a pump assembly operatively coupled to the elongate member via a tubular member, the tubular member being configured to house fluid, the pump assembly being configured to transfer fluid from the tubular member to the inflatable portion of the elongate member.

17. The implantable inflatable device of claim 16, wherein the tubular member includes a first portion having a first size and a second portion having a second size, the second size being larger than the first size.

18. The implantable inflatable device of claim 16, wherein the tubular member includes a first portion having a first size, a second portion having a second size, and a third portion having a third size, the second size being larger than the first size and larger than the third size, the second portion being disposed between the first portion and the third portion.

19. The implantable inflatable device of claim 16, wherein the elongate member is a first elongate member, the device further comprising:
a second elongate member.

20. The implantable inflatable device of claim 16, wherein the pump assembly is configured to be wirelessly coupled to a controller.
